(19) United States
(12) Patent Application Publication
(10) Pub. No.: US 2018/0263524 A1
(43) Pub. Date: Sep. 20, 2018

(54) AVOIDING MRI-INTERFERENCE WITH CO-EXISTING SYSTEMS

(71) Applicants: Shuki Vitek, Haifa (IL); Benny Assif, Ramat Hasharon (IL); Adi Greenberg, Haifa (IL)

(72) Inventors: Shuki Vitek, Haifa (IL); Benny Assif, Ramat Hasharon (IL); Adi Greenberg, Haifa (IL)

(21) Appl. No.: 15/979,602

(22) Filed: May 15, 2018

Related U.S. Application Data
(63) Continuation of application No. 13/222,086, filed on Aug. 31, 2011, now abandoned.

(51) Int. Cl. A61B 5/055; A61N 7/02; A61B 90/00; A61B 8/08; A61N 7/00; A61B 17/22

(52) U.S. Cl. CPC A61B 5/055; A61N 7/02; A61B 2090/374; A61B 2017/22007; A61B 2090/378; A61N 2007/0078; A61B 8/0808

(57) ABSTRACT

MRI interference with a co-existing treatment system may be reduced or avoided by carrying out RF-sensitive operations of the treatment system only when gradient field activity of the MRI system is suppressed.

### CROSS-REFERENCE TO RELATED APPLICATION

This is a continuation of U.S. patent application Ser. No. 13/222,086, filed on Aug. 31, 2011, the entire disclosure of which is hereby incorporated by reference.

### FIELD OF THE INVENTION

The present invention relates, generally, to medical diagnosis and treatment methods guided by magnetic resonance imaging (MRI), and, more specifically, to approaches to minimizing MRI-induced interferences.

### BACKGROUND

Magnetic resonance imaging may be used in conjunction with ultrasound focusing in a variety of medical applications. Ultrasound penetrates well through soft tissues and, due to its short wavelengths, can be focused to spots with dimensions of a few millimeters. As a consequence of these properties, ultrasound can be and has been used for various diagnostic and therapeutic medical purposes, including ultrasound imaging and non-invasive surgery. For example, focused ultrasound may be used to ablate diseased (e.g., cancerous) tissue without causing significant damage to surrounding healthy tissue. An ultrasound focusing system generally utilizes an acoustic transducer surface, or an array of transducer surfaces, to generate an ultrasound beam. In transducer arrays, the individual surfaces, or “elements,” are typically individually controllable, i.e., their vibration phases and/or amplitudes can be set independently of one another, allowing the beam to be steered in a desired direction and focused at a desired distance. The ultrasound system often also includes receiving elements, integrated into the transducer array or provided in form of a separate detector, that help monitor the focused ultrasound treatment, primarily for safety purposes. For example, the receiving elements may serve to detect ultrasound reflected off interfaces between the transducer and the target tissue, which may result from air bubbles on the skin that need to be removed to avoid skin burns. The receiving elements may also be used to detect cavitation in overheated tissues (i.e., the formation of cavities due to the collapse of bubbles formed in the liquid of the tissue).

To visualize the target tissue and guide the ultrasound focus during therapy, magnetic resonance imaging may be used. In brief, MRI involves placing a subject, such as the patient, into a homogeneous static magnetic field, thus aligning the spins of hydrogen nuclei in the tissue. Then, by applying a radio-frequency (RF) electromagnetic pulse of the right frequency (the “resonance frequency”), the spins may be flipped, temporarily destroying the alignment and inducing a response signal. Different tissues produce different response signals, resulting in a contrast among theses tissues in MR images. Because the resonance frequency and the frequency of the response signal depend on the magnetic field strength, the origin and frequency of the response signal can be controlled by superposing magnetic gradient fields onto the homogeneous field to render the field strength dependent on position. By using time-variable gradient fields, MRI “scans” of the tissue can be obtained. Many MRI protocols utilize time-dependent gradients in two or three mutually perpendicular directions. The relative strengths and timing of the gradient fields and RF pulses are specified in a pulse sequence and may illustrated in a pulse sequence diagram.

Time-dependent magnetic field gradients may be exploited, in combination with the tissue dependence of the MRI response signal, to visualize, for example, a brain tumor, and determine its location relative to the patient's skull. An ultrasound transducer system, such as an array of transducers attached to a housing, may then be placed on the patient's head. The ultrasound transducer may include MR tracking coils or other markers that enable determining its position and orientation relative to the target tissue in the MR image. Based on computations of the required transducer element phases and amplitudes, the transducer array is then driven so as to focus ultrasound into the tumor. Alternatively or additionally, the ultrasound focus itself may be visualized, using a technique such as thermal MRI or acoustic resonance force imaging (ARFI), and such measurement of the focus location may be used to adjust the focus position. These methods are generally referred to as magnetic-resonance-guided focusing of ultrasound (MRgFUS).

The simultaneous operation of ultrasound and MRI apparatus can lead to undesired interferences. For example, MRI is very sensitive to radio-frequency (RF) noise generated by the focused ultrasound system (see, e.g., U.S. Pat. No. 6,735,461). Conversely, focused ultrasound procedures often involve RF-sensitive operations (such as the ultrasound detection that may accompany treatment with focused ultrasound) that are easily disturbed by RF excitation signals and/or time-varying field gradient generated by the MRI system. Prior-art approaches to avoiding such interference include shielding as well as signal filtering and/or processing. Shielding the ultrasound system from interfering MR signals typically requires covering or surrounding the whole transducer and associated cables in metallic shield. In some systems, however, acoustic constraints prevent complete encapsulation of the ultrasound-receiving elements, resulting in penetration of, e.g., the front layer of a receiver and/or the cables by some amount of RF noise. Filtering unwanted RF disturbances from desired RF signals requires sophisticated electronics that is often difficult to implement and might damage the wanted signal. Digital signal processing usually increases the system complexity significantly, and is sometimes insufficient to eliminate all interferences. Accordingly, there is a need for alternative approaches in MRgFUS applications to minimize or avoid interferences between the two systems.

### SUMMARY

Embodiments of the present invention reduce or eliminate MRI interference with a co-existing system by exploiting MRI pulse sequences (also called “MRI recipes”) that include periods when the MRI gradients are relatively inactive (or “quiet”). The co-existing system may be a treatment system such as, for example, an ultrasound imaging probe or phased-array ultrasound transducer system. The operating procedure of the co-existing system may be synchronized with the MRI recipe such that RF-sensitive operations are carried out only during time intervals when the MRI gradients are inactive (and which are typically also free of MR excitation or response signals). Inactive gradients include gradients that are substantially zero, and may further include non-zero, but temporarily constant (or “static”) gradients. In practice, gradients are characterized as inactive if the RF noise that they generate is below a predetermined maximum acceptable noise limit, which generally depends on the particular application.

Avoidance of MRI-caused interference with ultrasound operations in accordance herewith is advantageous in that it generally eliminates (or at least reduces) the need for shielding, filtering, or digital signal processing of RF signals. Various embodiments of the present invention avoid the drawbacks of the prior art by confining the RF-receiving periods of the ultrasound system to time intervals in which there is no interference from MRI that would have to be shielded, filtered, or removed by post-processing. As a result, however, the total imaging or treatment time may be slightly increased. Therefore, it may be desirable for certain applications to combine the synchronization of RF-sensitive ultrasound operations and MRI gradient idle times with shielding, filtering, and/or signal processing to optimize the overall effectiveness of the MRgFUS system.

In a first aspect, the invention provides a method of performing treatment of an anatomic region in conjunction with MR imaging of the region, where the treatment includes at least one RF-sensitive operation. The RF-sensitive operation may be, for example, an ultrasound operation, which may include or consist of a cavitation or acoustic-reflection measurement or ultrasound imaging. The method involves temporarily suppressing gradient field activity during an MR imaging operation, and carrying out the RF-sensitive operation only when the gradient field activity is suppressed. Non-RF-sensitive treatment operations may be carried out while the gradient fields are active.

In some embodiments, gradient-field-activity suppression corresponds to substantially constant gradient fields, i.e., gradient fields whose magnitude changes by less than a predetermined fraction or absolute value. For example, in certain embodiments, gradient fields are deemed “substantially constant” if their magnitude changes, at a given point in time, by less than 0.1% of their maximum change rate.

The method may further include signaling onset of the gradient-field-activity suppression by an MRI apparatus (e.g., to an apparatus performing the treatment). In some embodiments, the MR imaging conforms to a pulse sequence that specifies the onset time of the gradient-field-activity suppression; the RF-sensitive operation may begin based on this onset time. During the pulse sequence, the gradient field activity may be suppressed periodically. The pulse sequence may have an associated repetition time period. The method may include determining the end of such repetition time period, carrying out the RF-sensitive operation after the repetition time period has ended, and triggering a new repetition time period after completion of the RF-sensitive operation. In some embodiments, the method includes synchronizing the treatment and the MR imaging with a synchronization signal. Alternatively or additionally, the treatment and the MR imaging may be synchronized to a common clock.

In another aspect, the invention provides a system for performing treatment of an anatomic region in conjunction with MR imaging of the region, where the treatment includes at least one RF-sensitive operation. The system includes an MRI apparatus for imaging the anatomic region (which involves gradient field activity), and a treatment controller (e.g., a controller associated with or part of the treatment system) in communication with the MRI apparatus. The treatment controller causes the RF-sensitive operation to be carried out only when the gradient field activity is suppressed. The system may further include an MRI controller for operating the MRI apparatus in accordance with a pulse sequence. In some embodiments, the MRI controller signals time intervals of the pulse sequence where the gradient field activity is suppressed to the treatment controller, such that the RF-sensitive operation is only performed during these time intervals In some embodiments, the treatment controller causes performance of the RF-sensitive operation when the pulse sequence ends, and triggers repetition of the pulse sequence after completion of the RF-sensitive operation. The system may further include the treatment apparatus (which may be, e.g., an ultrasound transducer) that performs the treatment.

In yet another aspect, a controller for synchronizing an MRI apparatus with a treatment system (such as an ultrasound system) is provided. The controller includes a module for receiving information about an MRI pulse sequence specifying time intervals wherein gradient fields are suppressed, and a module for initiating the RF-sensitive ultrasound operation at the onset of the gradient-field suppression based on the information.

A further aspect of the invention is directed to an MRI system operable in conjunction with a treatment system for performing MR imaging of an anatomic region in conjunction with treatment of the region (which includes one ore more RF-sensitive operations). The MRI system includes an MRI apparatus for imaging the anatomic region and an MRI controller. The MRI controller operates the MRI apparatus in accordance with a pulse sequence that includes time intervals of gradient field activity as well as time intervals where the gradient field activity is suppressed. The controller signals the time intervals where the gradient field activity is suppressed to the treatment apparatus so as to cause performance of the RF-sensitive operation during these time intervals.

Another aspect is directed to a treatment system operable in conjunction with an MRI system for performing treatment (including RF-sensitive operations) of an anatomic region in conjunction with MR imaging of the region. The system includes a treatment apparatus (such as, or including, an ultrasound transducer) for performing the treatment, and treatment controller for causing performance of the RF-sensitive operation in response to an end of an MRI pulse sequence comprising gradient field activity, and triggering repetition of the pulse sequence after completion of the RF-sensitive operation.

### BRIEF DESCRIPTION OF THE DRAWINGS

The foregoing will be more readily understood from the following detailed description, in particular, when taken in conjunction with the drawings, in which:

FIG. 1 is a schematic drawing of an MRgFUS system in accordance with one embodiment;

FIG. 2 is a perspective view of an MRgFUS system in accordance with one embodiment;

FIGS. 3A-3C are schematic drawings illustrating the interaction between an MRI apparatus and an ultrasound transducer in accordance with various embodiments;

FIG. 4 is a pulse sequence diagram illustrating an exemplary MRI protocol as well as synchronization-signal and ultrasound-detection periods in accordance with one embodiment;

FIG. 5A is a spectrum of an MRI interference signals detected by the ultrasound receiver in the absence of synchronization; and

FIG. 5B is a spectrum of an MRI interference signal detected by the ultrasound receiver after synchronization in accordance with one embodiment.

### DETAILED DESCRIPTION

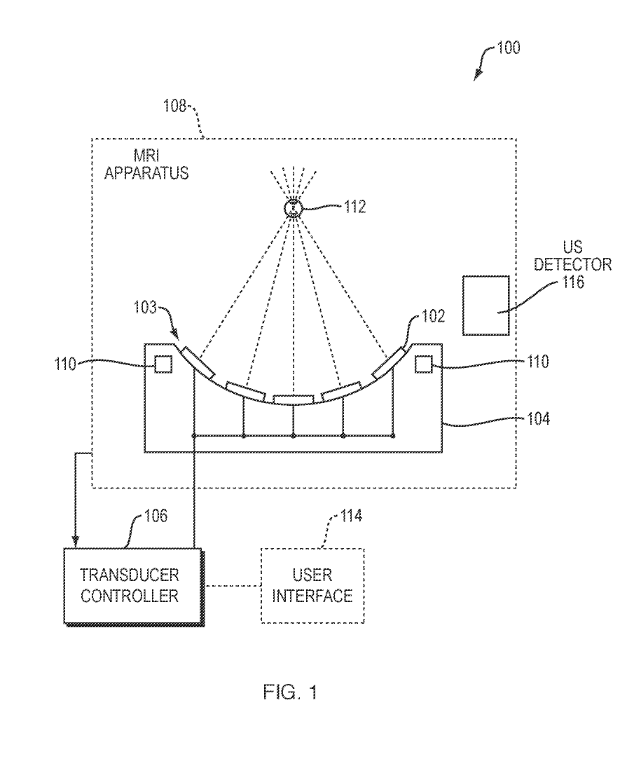

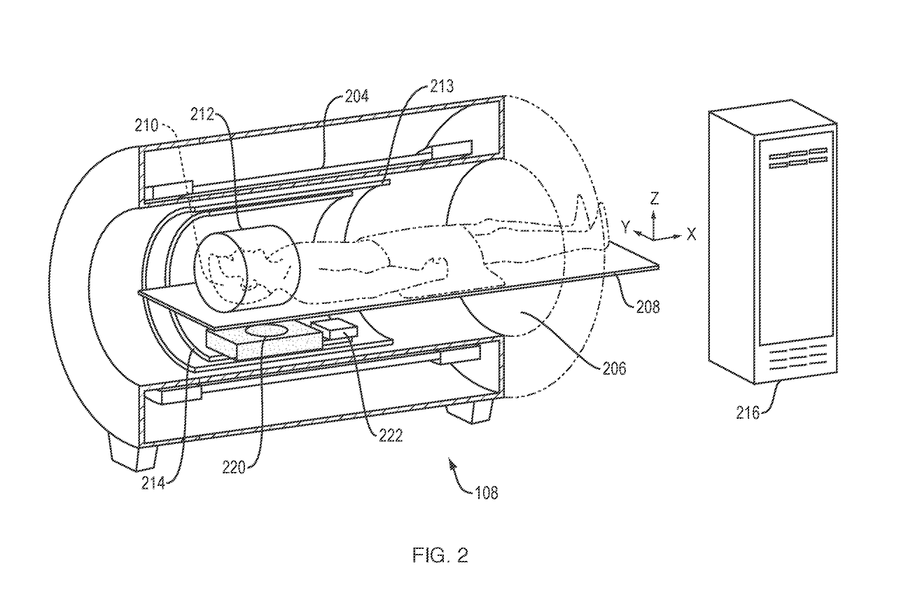

FIGS. 1 and 2 illustrate an exemplary MRgFUS system 100 in which synchronization of ultrasound and MRI procedures may advantageously be practiced. As shown in FIG. 1, the system 100 includes a plurality of ultrasound transducer elements 102, which are arranged in an array 103 at the surface of a housing 104. The array may comprise a single row or a matrix of transducer elements 102. In alternative embodiments, the transducer elements 102 may be arranged without coordination, i.e., they need not be spaced regularly or arranged in a regular pattern. The array may have a curved (e.g., spherical or parabolic) shape, as illustrated, or may include one or more planar or otherwise shaped sections. Its dimensions may vary, depending on the application, between millimeters and tens of centimeters. The transducer elements 102 may be piezoelectric ceramic elements. Piezo-composite materials, or generally any materials capable of converting electrical energy to acoustic energy, may also be used. To damp the mechanical coupling between the elements 102, they may be mounted on the housing using silicone rubber or any other suitable damping material.

The transducer elements 102 are separately controllable, i.e., they are each capable of emitting ultrasound waves at amplitudes and/or phases that are independent of the amplitudes and/or phases of the other transducers. A transducer controller 106 serves to drive the transducer elements 102. For n transducer elements, the controller 106 may contain n control circuits each comprising an amplifier and a phase delay circuit, each control circuit driving one of the transducer elements. The controller 106 may split an RF input signal, typically in the range from 0.1 MHz to 4 MHz, into n channels for the n control circuit. It may be configured to drive the individual transducer elements 102 of the array at the same frequency, but at different phases and different amplitudes so that they collectively produce a focused ultrasound beam. The transducer controller 106 desirably provides computational functionality, which may be implemented in software, hardware, firmware, hardwiring, or any combination thereof, to compute the required phases and amplitudes for a desired focus location. In general, the controller 106 may include several separable apparatus, such as a frequency generator, a beamformer containing the amplifier and phase delay circuitry, and a computer (e.g., a general-purpose computer) performing the computations and communicating the phases and amplitudes for the individual transducer elements 102 to the beamformer. Such systems are readily available or can be implemented without undue experimentation.

The system 100 further includes an MRI apparatus 108 for imaging the target tissue and/or ultrasound focus. To aid in determining the relative position of transducer array and MRI apparatus 108, the transducer array may have MR trackers 110 associated with it, arranged at a fixed position and orientation relative to the array. The trackers 110 may, for example, be incorporated into or attached to the housing 104. If the relative positions and orientations of the MR trackers 110 and transducers 102 are known, MR scans of the MR trackers 110 implicitly reveal the transducer location in MRI coordinates, i.e., in the coordinate system of the MRI apparatus 108. The transducer controller 106, which receives MRI data containing the MR tracker location, can then set the phases and amplitudes of the transducers 102 to generate a focus 112 at a desired location or within a desired target region. In some embodiments, a user interface 114 in communication with the transducer controller 106 and/or the MRI apparatus 108 facilitates the selection of the focus location or region in MR coordinates.

The system 100 generally also has the capability to detect ultrasound, which serves to monitor the application of ultrasound for safety purposes. For example, ultrasound reflections off tissue interfaces that intersect the ultrasound beam path may be analyzed to ensure, if necessary by adjustment of the treatment protocol, that such interfaces are not inadvertently overheated. Further, measurements of the received cavitation spectrum may be used to detect cavitation resulting from the interaction of ultrasound energy with water-containing tissue. In addition, the visualization of the tissue and target may be supplemented by ultrasound imaging, for example, to facilitate tracking a moving target. Ultrasound detection may be accomplished with the ultrasound transducer array 103. For example, treatment and imaging periods may be interleaved, or a contiguous portion of the array 103 or discontiguous subset of transducer elements 102 may be dedicated to imaging while the remainder of the array 103 focuses ultrasound for treatment purposes. Alternatively, a separate ultrasound receiver 116, which may be, e.g., a simple ultrasound probe or array of elements, may be provided. The separate receiver 116 may be placed in the vicinity of the ultrasound transducer array 103, or even integrated into its housing 104. If synchronization in accordance herewith is not utilized, the ultrasound receiver 116 needs to be shielded, e.g., by a surrounding conductive structure serving as a Faraday cage, to be at least partially effective.

FIG. 2 illustrates the MRI apparatus 108 in more detail. The apparatus 108 may include a cylindrical electromagnet 204, which generates the requisite static magnetic field within a bore 206 of the electromagnet 204. During medical procedures, a patient is placed inside the bore 206 on a movable support table 208. A region of interest 210 within the patient (e.g., the patient's head) may be positioned within an imaging region 212 wherein the electromagnet 204 generates a substantially homogeneous field. A set of cylindrical magnet field gradient coils 213 may also be provided within the bore 206 and surrounding the patient. The gradient coils 213 generate magnetic field gradients of predetermined magnitudes, at predetermined times, and in three mutually orthogonal directions. With the field gradients, different spatial locations can be associated with different precession frequencies, thereby giving an MR image its spatial resolution. An RF transmitter coil 214 surrounding the imaging region 212 emits RF pulses into the imaging region 212, and receives MR response signals emitted from the region of interest 210. (Alternatively, separate MR transmitter and receiver coils may be used.)

The MRI apparatus 108 generally includes an MRI controller 216 that controls the pulse sequence, i.e., the relative timing and strengths of the magnetic field gradients and the RF excitation pulses and response detection periods. The MRI controller 216 may be combined with the transducer controller 106 into an integrated system control facility. The MR response signals are amplified, conditioned, and digitized into raw data using an image processing system, and further transformed into arrays of image data by methods known to those of ordinary skill in the art. Based on the image data, a treatment region (e.g., a tumor) is identified. The image processing system may be part of the MRI controller 216, or may be a separate device (e.g., a general-purpose computer containing image processing software) in communication with the MRI controller 216 and/or the transducer controller 106. An ultrasound phased array 220, disposed within the bore 206 of the MRI apparatus and, in some embodiments, within the imaging region 212, is then driven so as to focus ultrasound into the treatment region. The drive signals are based on the MRI images, which provide information about the position and orientation of the transducer surface(s) with respect to the MRI apparatus and/or the focus location. To monitor the ultrasound treatment, an ultrasound receiver 222 may also be disposed within the bore 206 of the MRI apparatus.

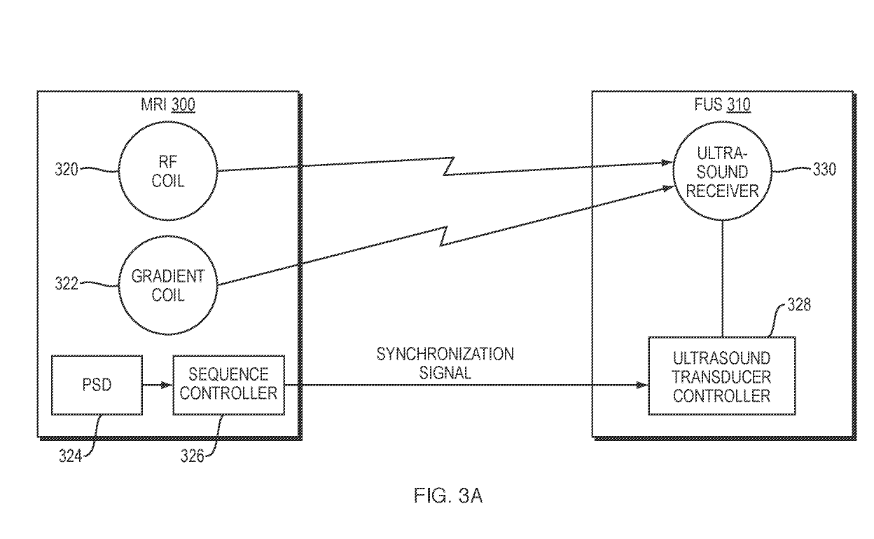

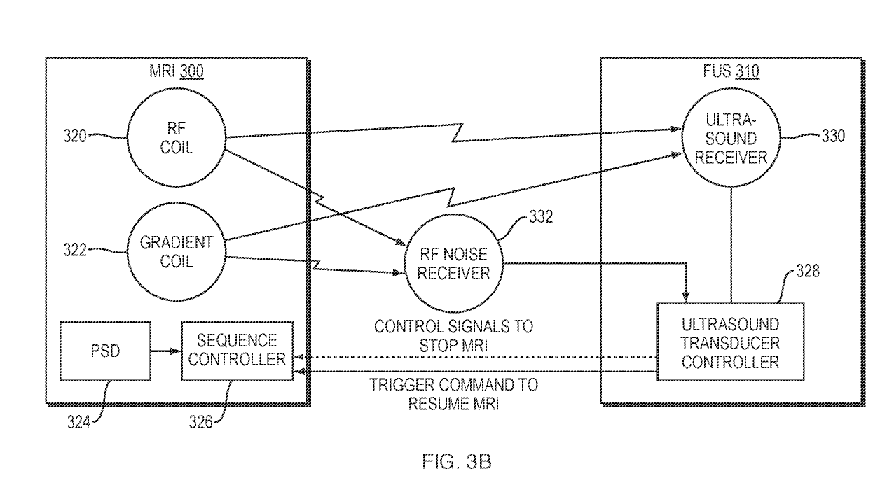

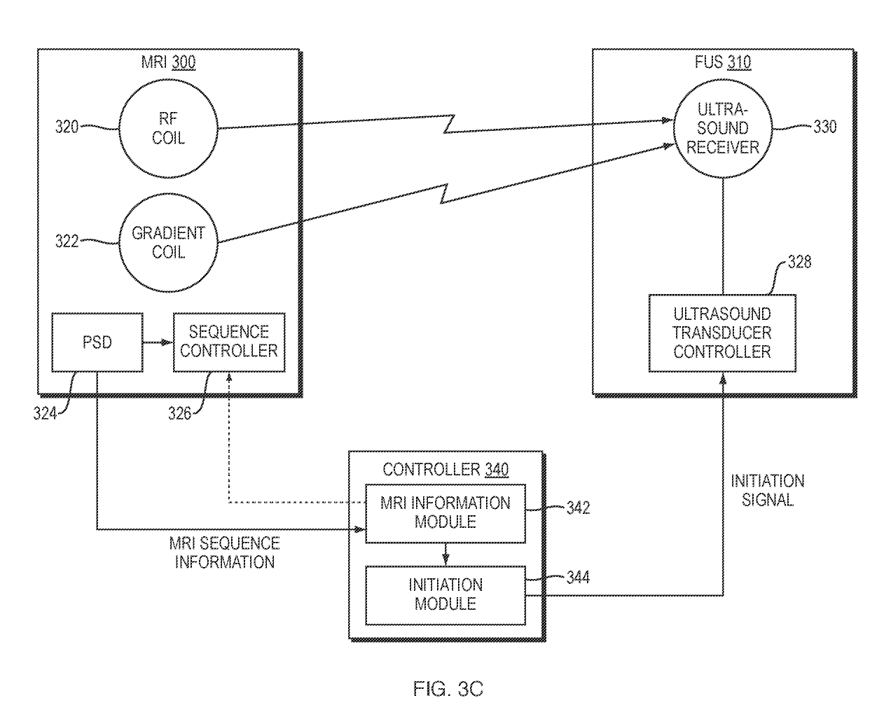

FIGS. 3A-3C schematically illustrates the interaction between an MRI apparatus 300 and a focused ultrasound system 310 in accordance with various embodiments of the invention. The MRI apparatus 300 includes RF transmitter coils 320, and gradient coils 322 for generating time-varying magnetic gradients across the tissue to be imaged. Both transmitter-coil and gradient-coil emissions fall in the RF range and can potentially disturb focused ultrasound procedures. The MRI transmitter coils 320 generate electromagnetic pulses with frequencies in the range from about 50 MHz to about 150 MHz to induce spin flipping. The gradients generated by the gradient coils 322 are typically updated at kHz or MHz frequencies, and are substantially constant between successive updates. For example, the gradient value (i.e., the magnetic field strength of the gradient field) may be controlled digitally at a sampling rate of 250kHz by applying a new voltage every four microseconds. These small control steps generate RF noise, mainly at the sampling frequency (i.e., 250 kHz in the example) and its harmonics (i.e., 500 kHz, 750 kHz, etc.). A step in the gradient value is usually implemented by a controlled ramp whose slope is proportional to the voltage step. The resulting RF noise is generally proportional to the voltage step as well. However, even during nominally static gradients, control steps exist and resulting in some level of RF noise (although significantly less than is generated during ramps). In other words, non-zero static gradients are quieter than dynamic gradients, but are not completely quiet.

The MRI apparatus 300 includes a database 324 (stored, e.g., on a hard drive of a computer, which may be the same computer as is used for MR image processing) for storing pulse sequence diagrams (PSDs). An associated sequence controller 326 within the MRI controller 216 operates the MRI apparatus in accordance with the specified pulse sequences. As illustrated in FIG. 3A, the sequence controller 326 may provide a synchronization signal to the ultrasound control module 328, signaling the onset of gradient idle times, i.e., time intervals in which magnetic field gradients, or time variations thereof, are completely or partially suppressed. The ultrasound controller module 328 may be implemented in the transducer controller 106, and initiate RF-sensitive ultrasound operations only during the gradient idle times.

Ultrasound operations that are particularly sensitive to RF disturbances from the MRI apparatus 300 include ultrasound imaging (in parallel with MRI) and measurements of the cavitation spectrum or of acoustic reflections, all of which generally have low signal voltages associated with them (e.g., voltages in the mV range and below). During these measurements, the ultrasound receiver 330 (which may be the transducer operated in “listening” mode, or a separate, dedicated receiver device) converts the acoustic signals into electrical RF signals. Such signals can also be created by the RF disturbances from the MRI apparatus 300, resulting in unwanted signal components. Since the detected signals generally have lower power than, e.g., focused ultrasound ablation pulses, they are particularly sensitive to such perturbations.

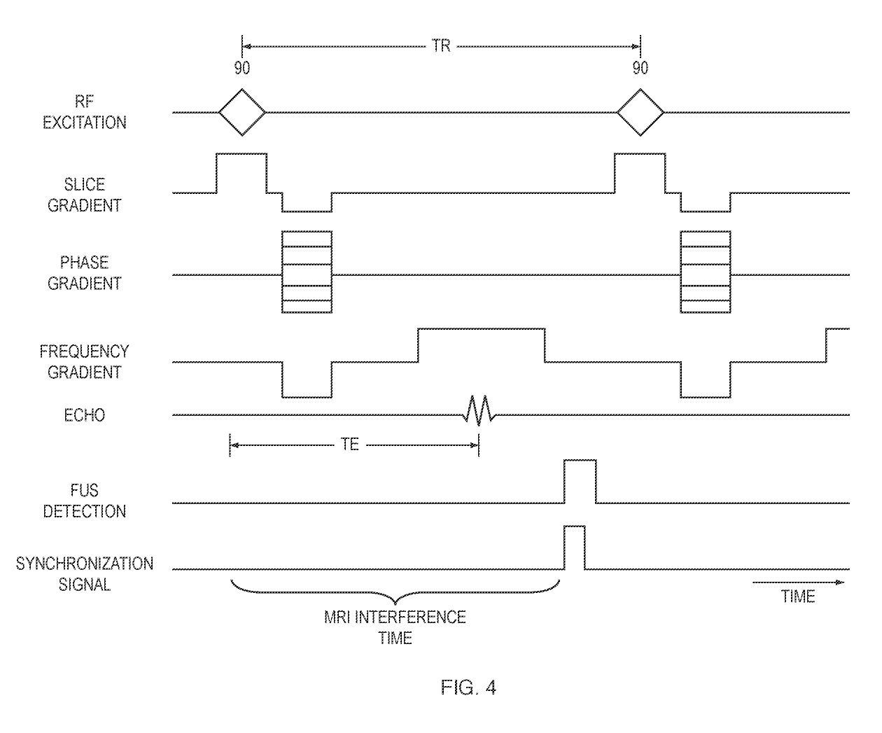

FIG. 4 shows a PSD illustrating, for a typical MR gradient echo pulse sequence, the relative timing of the RF excitation pulse, the magnetic field gradients in three directions, and the MR response signal, which occurs at the echo time (TE). The sequence may be periodically repeated; the period is denoted as the repetition time TR and may be, for example, in the range from 20 to 30 ms. FIG. 4 further shows the timing of the synchronization signal relative to the MRI sequence, as well as the period during which RF-sensitive ultrasound operations (i.e., generally, ultrasound detection) may be carried out, which may last, for example, 1 ms. Ultrasound operations that are not especially sensitive to RF disturbances may be carried out at any time, including periods during which the MRI gradients are active. In fact, focused ultrasound application times are often in the range from about 10 to about 30 seconds. Thus, ultrasound application may begin long before and end long after the MRI sequence. In embodiments in which the ultrasound transducer is alternately used for focused ultrasound application and RF-sensitive ultrasound detection, the non-RF-sensitive operations are preferably carried out during active-gradient periods, reserving the gradient idle times for RF-sensitive operations.

In the PSD shown in FIG. 4, gradient idle time is added to each repetition time period by design (in a first step of a first embodiment). During the idle time, the MRI apparatus 300 sends a synchronization signal to the ultrasound system 310 (in a second step of a first embodiment), which then performs spectrum measurements and other RF-sensitive operations (in a third step of a first embodiment). Synchronization between MRI and ultrasound detection is, thus, internally controlled by the MRI recipe. In alternative embodiments, synchronization may be effected through control mechanisms external to the MRI recipe. For example, as illustrated in FIG. 3B, the ultrasound controller module 328 may control the timing of RF-sensitive operations based on measurements of RF signals originating from the MRI apparatus 300, which may be performed, for example, by the ultrasound receiver 330 or by a separate, dedicated RF-noise receiver 332 in communication with the module 328. The MRI sequence may stop running after each repetition period (either automatically or based on an external control signal) (in a first step of a second embodiment), and the focused ultrasound system may identify the end of a repetition period (e.g., by measuring RF signals generated by the MRI apparatus) (in a second step of a second embodiment), perform the ultrasound measurements (in a third step of a second embodiment), and then send a trigger command to the MRI apparatus to resume the MRI sequence, i.e., proceed to the next repetition period (in a fourth step of a second embodiment). The MRI sequence may be interrupted after each repetition time, or after a multiple of the repetition time (such that one or more repetition periods are skipped before the next ultrasound measurement is carried out). The system may also be programmed to perform ultrasound measurements only after certain MRI procedures, for example, only after thermal imaging sequences. External control generally provides a high degree of flexibility in timing MR imaging and RF-sensitive ultrasound operations, thereby facilitating time efficiency in the overall procedure.

The synchronization of the MRI and focused ultrasound apparatus 300, 310 may be modified in additional ways. For example, the sequence controller 326 and ultrasound controller module 328 may be integrated into a single control module that sends synchronization or clock signals simultaneously to both apparatus 300, 310, or controls the MRI transmitter coils 320, gradient coils 322, and ultrasound receiver 330 directly. Alternatively, as shown in FIG. 3C, a separate controller 340 may communicate with conventional MRI and ultrasound apparatus that each include their individual controllers. The controller 340 may include a first module 342 that determines when gradient-field activity is suppressed, e.g., based on information it receives about an MRI pulse sequence specifying time intervals during which the gradients are quiet. The module 342 may also send control signals to the sequence controller 326 to stop MRI operation at the end of a sequence. The first module 342 may communicate gradient idle time to a second module 344 responsible for initiating the RF-sensitive treatment operation.

In general, functionality for synchronizing an MRI apparatus and a focused ultrasound system as described above, whether integrated with the MRI and/or ultrasound controller or provided by a separate controller, may be structured in one or more modules implemented in hardware, software, or a combination of both. For embodiments in which the functions are provided as one or more software programs, the programs may be written in any of a number of high level languages such as FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80x86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

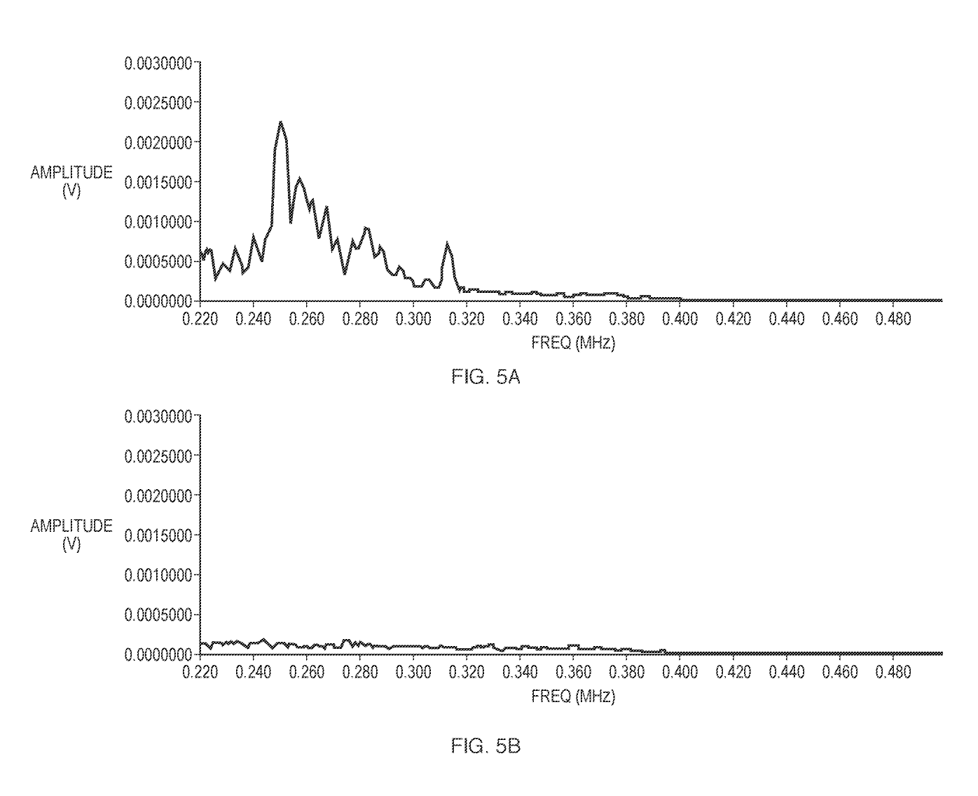

FIGS. 5A and 5B show the spectra of MRI interference signals detected by the ultrasound transducer with and without synchronization of the detection period with gradient idle times. As illustrated, synchronization can reduce MRI disturbances by about an order of magnitude. Note that the signals in FIGS. 5A and 5B are free of cavitation effects. FIG. 5A shows a signal corrupted by gradient noise, while FIG. 5B, shows a clean signal that contains only background noise.

In some embodiments, the synchronization methods described above are used in conjunction with shielding, signal filtering, and/or processing. This allows RF-sensitive operations to be carried out during portions of MR sequences in which the gradients are sufficiently inactive. For example, if synchronization is combined with shielding, there is generally a trade-off between the amount of shielding used and the maximum acceptable noise. The less shielding is used, the quieter the gradients need to be to avoid undesired interference between the MRI system and the ultrasound (or other co-existing) system. Noise reductions due to shielding depend on the particular material used (e.g., iron, copper, or nickel) as well as on the frequency range of interest, and can readily be ascertained based on graphs and tabulations of absorption and reflection coefficients that are available in the literature. For example, at frequencies of around 1 MHz, a 3 mm thick iron shield reduces the noise by about 100 dB. For a given maximum acceptable noise level (which, in turn, depends on the signal filtering and processing capabilities of the system), the maximum allowable gradients can be computed based on the noise reduction achieved by shielding.

Although the present invention has been described with reference to an ultrasound transducer system and other specific details, it is not intended that such details should be regarded as limitations upon the scope of the invention. For example, systems and methods for synchronizing MR imaging with treatment modalities other than focused ultrasound therapy that include RF-sensitive operations are also included within the scope of the invention. Moreover, it is to be understood that the features of the various embodiments described herein are not necessarily mutually exclusive and can exist in various combinations and permutations, even if such combinations or permutations are not made express herein, without departing from the spirit and scope of the invention. In fact, variations, modifications, and other implementations of what is described herein will occur to those of ordinary skill in the art without departing from the spirit and the scope of the invention.

What is claimed is:

1. A system for performing treatment of an anatomic region in conjunction with MR imaging of the region, the treatment comprising at least one RF-sensitive operation, the system comprising:
an MRI apparatus for imaging the anatomic region, the imaging comprising gradient field activity; and
a treatment system comprising a treatment controller in communication with the MRI apparatus, the controller being configured to:
(i) cause the MRI apparatus to temporarily suppress the gradient field activity during imaging of the anatomic region;
(ii) receive a signal indicative of gradient-field-activity suppression; and
(iii) initiate the RF-sensitive operation in response to the received signal indicative of the gradient-field-activity suppression.

2. The system of claim 1, further comprising an MRI controller for operating the MRI apparatus in accordance with a pulse sequence that includes time intervals where the gradient field activity is suppressed, the signal being indicative of the time intervals.

3. The system of claim 1, further comprising an MRI controller for operating the MRI apparatus in accordance with a pulse sequence, the signal being indicative of an end of the pulse sequence.

4. The system of claim 3, wherein the treatment controller is further configured to trigger repetition of the pulse sequence so as to allow the MRI controller to operate the MRI apparatus in accordance therewith after completion of the RF-sensitive operation.

5. The system of claim 1, further comprising a measurement system for measuring RF signals originating from the MRI apparatus, wherein the controller is further configured to perform the RF-sensitive operation based on the measured RF signals.

6. The system of claim 1, wherein the MRI apparatus comprises an MRI controller for transmitting the signal indicative of gradient-field-activity suppression to the treatment controller.

7. The system of claim 1, wherein the treatment system further comprises at least one of an ultrasound transducer, a cavitation detection device, or a reflection detection device, in communication with the treatment controller, for performing the RF-sensitive operation.

8. The system of claim 1, wherein the treatment controller is further configured to cause the gradient field activity to be periodically temporarily suppressed.

9. The system of claim 1, further comprising a system clock for synchronizing the RF-sensitive operation and imaging of the anatomic region.

10. A method of performing treatment of an anatomic region in conjunction with magnetic resonance (MR) imaging of the region, the treatment comprising at least one radio-frequency-sensitive (RF-sensitive) operation, the method comprising the steps of:
(i) during an MR imaging operation, temporarily suppressing gradient field activity; and
(ii) in response to a signal indicative of gradient-field-activity suppression, initiating the RF-sensitive operation.

11. The method of claim 10, wherein the gradient-field-activity suppression corresponds to zero gradient fields.

12. The method of claim 10, wherein the gradient-field-activity suppression corresponds to constant gradient fields.

13. The method of claim 10, wherein the signal indicative of gradient-field-activity suppression is transmitted from an MR imaging apparatus performing the MR imaging operation to an RF device performing the RF-sensitive operation.

14. The method of claim 10, wherein the MR imaging conforms to a pulse sequence specifying an onset time of the gradient-field-activity suppression, and the signal is indicative of the onset time.

15. The method of claim 14, wherein the gradient field activity is suppressed periodically during the pulse sequence.

16. The method of claim 10, wherein the MR imaging conforms to a pulse sequence having a repetition time period associated therewith, the signal indicating when the repetition time period ends.

17. The method of claim 16, further comprising triggering a new repetition time period after completion of the RF-sensitive operation.

18. The method of claim 10, wherein the signal is a synchronization signal specifying relative timing between the RF-sensitive operation and the MR imaging.

19. The method of claim 10, wherein the RF-sensitive operation and the MR imaging are synchronized to a common clock.

20. The method of claim 10, wherein the RF-sensitive operation is an ultrasound-detection operation.

21. The method of claim 20, wherein the ultrasound-detection operation comprises at least one of measuring cavitation, measuring acoustic reflections, or ultrasound imaging.

22. A controller for synchronizing an MRI apparatus performing an MR imaging operation with a treatment system performing operations at least one of which is RF-sensitive, the controller comprising:
a first module for (i) receiving information about an MRI pulse sequence specifying time intervals wherein gradient field activity is suppressed, and (ii) based thereon, determining when the gradient-field activity is suppressed; and
a second module for initiating the RF-sensitive operation at an onset of the gradient-field-activity suppression based on the determination.

23. An MRI system operable in conjunction with a treatment system for performing MR imaging of an anatomic region in conjunction with treatment of the region, the treatment comprising at least one RF-sensitive operation, the MRI system comprising:
an MRI apparatus for imaging the anatomic region, the imaging comprising gradient field activity;
a database for storing a pulse sequence comprising time intervals where the gradient field activity is suppressed; and
an MRI controller configured to:
(i) operate the MRI apparatus in accordance with the pulse sequence, and
(ii) signal the time intervals to the treatment system so as to cause performance of the RF-sensitive operation during the time intervals.

24. A treatment system operable in conjunction with an MRI system for performing treatment of an anatomic region in conjunction with MR imaging of the region, the treatment comprising at least one RF-sensitive operation, the treatment system comprising:
a treatment apparatus for performing the treatment; and
a treatment controller configured to:
(i) receive a signal indicative of an end of an MRI pulse sequence comprising gradient field activity,
(ii) in response to the received signal, initiate performance of the RF-sensitive operation; and
(iii) trigger repetition of the pulse sequence after completion of the RF-sensitive operation.

25. The treatment system of claim 24, wherein the treatment apparatus comprises an ultrasound transducer.
